(19) United States
(12) Patent Application Publication
(10) Pub. No.: US 2019/0269752 A1
(43) Pub. Date: Sep. 5, 2019

(54) Methods for the Prevention or Treatment of Epilepsy

(71) Applicant: Duke University, Durham, NC (US)

(72) Inventors: James O. McNamara, Durham, NC (US); Gumei Liu, Durham, NC (US); Bin Gu, Durham, NC (US); Xiao-Ping He, Durham, NC (US); Kamesh Krishnamurthy, Durham, NC (US); Yangzhong Huang, Durham, NC (US); Robert Mook, Durham, NC (US)

(21) Appl. No.: 16/282,139

(22) Filed: Feb. 21, 2019

Related U.S. Application Data
(60) Provisional application No. 62/633,516, filed on Feb. 21, 2018.

(51) Int. Cl. A61K 38/10; A61P 25/08; A61K 9/00

(52) U.S. Cl. CPC A61K 38/10; A61K 9/0019; A61P 25/08

(57) ABSTRACT

The present disclosure relates to methods of preventing or treating epilepsy comprising administering a receptor tyrosine kinase B (TrkB) inhibitor. In particular, the present disclosure relates to methods of treating a subject susceptible to the development of epilepsy, methods of inducing remission of epilepsy in a subject, and methods of transforming medically refractory epilepsy in a subject to medically responsive epilepsy comprising administering a therapeutically effective amount of a TrkB inhibitor or a phospholipase Cγ1 (PLCγ1) inhibitor.

### CROSS REFERENCE TO RELATED APPLICATIONS

This application claims priority to U.S. Provisional Patent Application Ser. No. 62/633,516, filed Feb. 21, 2018, the contents of which are hereby incorporated by reference in their entirety.

### STATEMENT REGARDING FEDERALLY SPONSORED RESEARCH OR DEVELOPMENT

This application was made with United States government support under Federal Grant No. NS056217 awarded by the NIH-NINDS. The United States government has certain rights in this invention.

### BACKGROUND OF THE INVENTION

### Field of the Invention

The present disclosure relates to methods for the prevention or treatment of epilepsy. More particularly, the present invention relates to methods of treating a subject susceptible to the development of epilepsy, methods of inducing remission of epilepsy, and methods of transforming medically refractory epilepsy in a subject to medically responsive epilepsy.

### Description of the Related Art

Temporal lobe epilepsy (TLE) is a common and commonly devastating form of human epilepsy. Despite the introduction of a panoply of new antiseizure drugs in the past quarter century, there has been no measurable improvement in the proportion of patients with newly diagnosed epilepsy rendered free of seizures (Chen et al 2017). Approximately one-third of such patients experience recurrent seizures despite treatment by skilled clinicians with recently introduced therapeutics (Chen et al 2017). The absence of preventive or disease modifying therapy for medically refractory temporal lobe epilepsy (TLE) poses a serious public health problem and great economic burden. These failures underscore the need to elucidate the mechanisms underlying the development and/or progression of epilepsy, a process termed epileptogenesis (Hauser 2018). Indeed, evidence from clinical and preclinical studies supports the idea that an episode of prolonged seizures (status epilepticus, SE) contributes to development of a form of medically refractory TLE. (Annegers et al 1987; Engel et al 1998; French et al 1993; Pitkanen et al 2010; Tsai et al 2009).

Clinical observations led Gowers (1881) to propose that seizures themselves could promote worsening of epilepsy. In support of this idea, longitudinal observations of a cohort of patients with newly diagnosed epilepsy revealed that individuals at low risk of recurrent seizures exhibited a progressive increase in risk with increasing numbers of seizures (Hauser and Lee 2002). Direct evidence that an isolated seizure could promote both development and progression of epilepsy emerged from preclinical observations (Goddard 1967). Repeatedly evoking brief, localized electrographic seizures induced a progressive increase in propagation and duration of evoked electrographic seizures accompanied by tonic-clonic behavioral seizures, a model termed kindling (Goddard 1967; Goddard et al 1969). Evoking many (e.g. 70-80) such seizures culminated in recurrent seizures occurring without stimulation, often associated with fatality (Pinel and Rovner 1978). Importantly, the frequency and severity of seizures progressively increases long after the onset of epilepsy in models induced by hypoxia-ischemia or status epilepticus; the occurrence of seizures is thought to contribute to this progression (Hellier et al 1998; Wiliams et al 2009).

In previous work, the inventors identified a molecular target that can prevent TLE in mice, namely the brain-derived neurotrophic factor (BDNF) receptor tyrosine kinase B, TrkB (Liu et al 2013), as well as the effector by which TrkB activation promotes development of epilepsy, namely the enzyme phospholipase Cγ1 (Gu et al 2015). One molecular consequence of either an isolated or repeated seizures is the increased activation of the BDNF receptor, TrkB, and its signaling effector, PLCγ1, spanning a couple of days as evident in biochemical and histochemical measures (Binder et al 1999; He et al 2010; Liu et al 2013; Gu et al 2015). Transient inhibition of TrkB-PLCγ1 signaling following status epilepticus prevents subsequent development of epilepsy (Liu et al 2013; Gu et al 2015). The selective inhibition of PLCγ1 following SE inhibited TLE yet preserved the neuroprotective effects of endogenous TrkB signaling.

### SUMMARY OF THE INVENTION

In a first aspect, the present invention provides a method of treating a subject susceptible to the development of epilepsy comprising administering to the subject a therapeutic amount of a receptor tyrosine kinase B (TrkB) inhibitor. In one embodiment, the treating comprises limiting the progression of epileptogenesis, and in another embodiment, the treating comprises the reversion of epileptogenesis to an earlier stage.

In a second aspect, the present invention provides a method of inducing remission of epilepsy in a subject comprising administering a therapeutically effective amount of a TrkB inhibitor or a PLCγ1 inhibitor.

In a third aspect, the present invention provides a method of transforming medically refractory epilepsy in a subject to medically responsive epilepsy comprising administering a therapeutically effective amount of a TrkB inhibitor or a PLCγ1 inhibitor, optionally in combination with one or more antiseizure drugs.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure.

FIG. 1A-FIG. 1D. Evoked seizure in kindled animals results in increased duration of subsequent electrographic and behavioral seizure. (FIG. 1A) Schematic of experimental design. Control animals from experiments depicted in FIGS. 2 & 3 were pooled for representation. (FIG. 1B-FIG. 1D) Electrographic seizure duration, behavioral seizure duration, and seizure score for animals receiving control treatment after an evoked seizure (n=28). Data was analyzed by two-way ANOVA with repeated measures and post-hoc Bonferroni test. *** p<0.001

FIG. 2A-FIG. 2D. Chemical-genetic inhibition of TrkB kinase after an evoked seizure reduces duration and severity of subsequent seizures. (FIG. 2A) Schematic of experimental design. (FIG. 2B-FIG. 2D) Electrographic seizure duration, behavioral seizure duration, and seizure score in animals receiving 1NMPP1 (n=16) or vehicle (n=15) following an evoked seizure. Data was analyzed by two-way ANOVA with repeated measures and post-hoc Bonferroni. * p<0.05, ** p<0.01, *** p<0.001

FIG. 3A-FIG. 3D. pY816 treatment after an evoked seizure reduces duration and severity of subsequent seizures. (FIG. 3A) Schematic of experimental design. (FIG. 3B-FIG. 3D) Electrographic seizure duration, behavioral seizure duration, and seizure score for animals receiving pY816 (n=17) or Scr (n=13) after an evoked seizure. Data was analyzed by two-way ANOVA with repeated measures and post-hoc Bonferroni test. * p<0.05, ** p<0.01, *** p<0.001

FIG. 4A-FIG. 4G. 1NMPP1 or pY816 treatment in the absence of a preceding evoked seizure has no effect on subsequent seizure severity. (FIG. 4A) Schematic of experimental design. (FIG. 4B-FIG. 4D) Electrographic seizure duration, behavioral seizure duration, and seizure score for animals receiving 1NMPP1 (n=10) or vehicle (n=10) in the absence of a preceding evoked seizure. (FIG. 4E-FIG. 4G). Electrographic seizure duration, behavioral seizure duration, and seizure score for animals receiving pY816 (n=16) or Scr (n=14) in the absence of a preceding evoked seizure. Data was analyzed by two-way ANOVA with repeated measures and post-hoc Bonferroni test.

FIG. 5A-FIG. 5D. Treatment with carbamazepine (CBZ) immediately after an evoked seizure has no effect on subsequent seizure class or duration. (FIG. 5A) Experimental design. (FIG. 5B-FIG. 5D) Electrographic seizure duration, behavioral seizure duration, and seizure score for animals receiving carbamazepine or vehicle following an evoked seizure. The short half-life of carbamazepine in mice necessitated treatment at four hour intervals which led us to treat all animals in a single experiment; as a result, the latency between the final kindled seizure and Post-Kindling Seizure #1 was variable in this group compared to experiments with 1NMPP1 and pY816. Data was analyzed by two-way ANOVA with repeated measures and post-hoc Bonferroni test.

FIG. 6. Schematic showing two major TrkB signaling pathways, SHC (Y515) and PLCγ (Y816).

FIG. 7A-FIG. 7B. Data showing a single intraperitoneal injection of pY816 peptide inhibited p-PLCγ1 (pY783) in adult mice.

FIG. 8. Schematic showing an experimental design for EEG and video monitoring following induction of status epilepticus. Following baseline recording of two weeks, animals were treated with either pY816 or a control twice daily for two weeks.

FIG. 9. Data that pY816 induces remission of seizures in epileptic animals.

FIG. 10A-FIG. 10B. Seizures recur upon discontinuation of antiseizure drug. Spontaneous recurrent seizures were detected in continuous video-EEG recordings performed in six animals commencing a few weeks following status epilepticus induced by infusion of kainic acid into the amygdala. (A) Trial design: the number of seizures were detected during two weeks baseline (prior to treatment), during two weeks on carbamazepine (Tx) (800 mg/kg/day in food pellets), and during two weeks following termination of treatment (Post-Tx). (B) The number of seizures detected in each animal (each designated by a unique symbol) recorded during baseline was reduced in each of the six animals during treatment with carbamazepine (Tx). Unlike treatment with pY816, seizures recurred in each animal following cessation of carbamazepine treatment.

### DETAILED DESCRIPTION OF THE INVENTION

Before the disclosed processes and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

It is also to be understood that unless clearly indicated otherwise by the context, embodiments disclosed for one aspect or embodiment of the invention can be used in other aspects or embodiments of the invention as well, and/or in combination with embodiments disclosed in the same or other aspects of the invention. Thus, the disclosure is intended to include, and the invention includes, such combinations, even where such combinations have not been explicitly delineated.

### Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a dose range is stated as 10-20 mg/kg, it is intended that values such as 10-15 mg/kg, 15-20 mg/kg, or 12-18 mg/kg, etc., are expressly enumerated in this specification. These are only examples of what is specifically intended, and all possible combinations of numerical values between and including the lowest value and the highest value enumerated are to be considered to be expressly stated in this disclosure. Additionally, the recitation of ranges is meant to include individual values within the range as if they were individually recited herein, for example if a dose range is stated as 10-20 mg/kg, it is intended that values such as 10 mg/kg, 15 mg/kg, 20 mg/kg, etc. are expressly enumerated in this specification.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the disease or disorder is epilepsy, and in other embodiments, it is temporal lobe epilepsy (TLE). In some embodiments, the TLE is medically refractory TLE. Thus, the terms “treatment” and “prevention” are used herein to refer to the limiting of or the prevention of the progression of epileptogenesis, or to the reversion of epileptogenesis to an earlier stage, including, e.g. the remission of TLE or transforming medically refactory to medically responsive TLE.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject is a human.

As used herein, a “subject susceptible to the development of epilepsy” is a subject undergoing epileptogenesis.

As used herein, the phrase “immediately following” with reference to a seizure means within about 30 minutes from the time of termination of the seizure.

Unless otherwise defined, all technical terms used herein have the same meaning as commonly understood by one of ordinary skill in the art to which this disclosure belongs.

### Limitation of the Progression of Epileptogenesis/Reversion of Epileptogenesis to an Earlier Stage

The inventors have surprisingly discovered that brief inhibition of TrkB-PLCγ1 signaling initiated after a seizure provides an approach to limiting seizure-induced progression of epileptogenesis. More specifically, the inventors have discovered that the inhibition of TrkB-PLCγ1 signaling commencing after an isolated seizure limits progression of epileptogenesis evidenced by the increased severity and duration of subsequent seizures. Moreover, the inventors discovered that fleeting inhibition of TrkB-PLCγ1 signaling initiated following a seizure reverted a subset of animals to an earlier state of epileptogenesis, while a similar treatment in the absence of a preceding seizure had no effect.

Accordingly, in a first aspect the present disclosure provides a method of treating a subject susceptible to the development of epilepsy comprising administering to the subject a therapeutic amount of a receptor tyrosine kinase B (TrkB) inhibitor. In one embodiment of this aspect of the invention, treating comprises limiting the progression of epileptogenesis, i.e. preventing the increased duration and severity of subsequent seizures, or where subsequent seizures do not occur. In another emodiment of this aspect of the invention, treating comprises the reversion of epileptogenesis to an earlier stage, i.e. providing for less severe seizures.

In some embodiments of the first aspect of the invention, the TrkB inhibitor is administered to the subject immediately following termination of an isolated seizure. By immediately following it is meant within about 30 minutes, which would include any time within 30 minutes (whether specifically enumerated or not), e.g. within about 25 minutes, within about 20 minutes, within about 15 minutes, within about 10 minutes, within about 5 minutes, or within about 1 minute. One of ordinary skill in the art will understand that in some instances the TrkB inhibitor may be administered at a time greater than about 30 minutes following termination of the seizure while still maintaining the effectiveness of administering the inhibitor. For example, in some instances the TrkB inhibitor may be administered to the subject within about 60 minutes of termination of an isolated seizure. Automated seizure detection systems may be used in conjunction with the methods disclosed herein to allow for notification of seizure activity and the need to introduce treatment in a timely manner, i.e. the methods of the invention may include a step wherein the subject is notified of a seizure (including the termination of the seizure) by a seizure detection system, after which the TrkB inhibitor is administered in accordance with the invention.

In certain embodiments of the first aspect of the invention, the isolated seizure is not status epilepticus (SE).

In one embodiment of the first aspect of the invention, the TrkB inhibitor is administered twice daily at a dose of about 10-20 mg/kg for a period of 2-3 days.

### Remission of TLE

The inventors have also surprisingly discovered that the short term administration of a TrkB inhibitor, in particular a PLCγ1 inhibitor, after onset of epilepsy can reduce the severity of the epilepsy and even induce remission, i.e. reduce the number of spontaneous recurrent seizures (SRS) or render the subject free of SRS following treatment. These findings of a reduction in the severity of epilepsy indicate that treatment as set forth herein would also provide an enhanced response to conventional antiseizure medications, including transforming medically refractory to medically responsive epilepsy.

Thus, in a second aspect of the invention, the present disclosure provides a method of inducing remission of epilepsy in a subject comprising administering a therapeutically effective amount of a TrkB inhibitor or a PLCγ1 inhibitor. In a third aspect, the invention provides a method of transforming medically refractory epilepsy in a subject to medically responsive epilepsy comprising administering a therapeutically effective amount of a TrkB inhibitor or a PLCγ1 inhibitor optionally in combination with one or more antiseizure drugs.

In these aspects of the invention, the subject may be suffering from partial epilepsy. In certain embodiments of these aspects of the invention, the subject is suffering from temporal lobe epilepsy (TLE), and in other embodiments, the TLE is medically refractory TLE.

In one embodiment of the second and third aspects of the invention, the inhibitor is administered twice daily at a dose of about 10-20 mg/kg for a period of about 2 weeks.

### TrkB Inhibitors

The TrkB inhibitors that may be used with any aspect of the present invention may be any compound that inhibits the activity of TrkB, including those known in the art. Exemplary TrkB inhibitors include, but are not limited to, larotrectinib, ANA 12, and Cyclotraxin B. Additional exemplary TrkB inhibitors include, but are not limited to, Altiratinib (DCC-2701, DCC270, DP5164), Belizatinib (TSR-011), Cabozantinib (XL-184, BMS-907351), Dovitinib (TKI-258, CHIR-258), DS-6051b, Entrectinib (RXDX-101), F17752, Loxo-101 (arry-470), Milciclib (PHA-848125AC), Sitravatinib (MGCD516), ASP7962, GZ38998, ONO-4474, and VM902A (Bailey et al 2017).

In some embodiments, the TrkB inhibitor prevents activation of phospholipase Cγ1 (PLCγ1) by TrkB and may be any such inhibitor known in the art. In some embodiments, the TrkB inhibitor is a PLCγ1 inhibitor. In some embodiments, the TrkB inhibitor is or comprises pY816.

In some embodiments, the PLCγ1 inhibitor binds the SH2 domain of PLCγ1, and prevents activation of PLCγ1 by receptor and non-receptor tyrosine kinases, including, but not limited to, TrkB. The PLCγ1 inhibitor may be any such inhibitor known in the art. In some embodiments, the PLCγ1 inhibitor is or comprises pY816 or a small molecule.

pY816 corresponds to amino acids 806-819 of human TrkB in which Y816 is phosphorylated, and wherein the protein transduction domain of the HIV-1 TAT protein is fused to the N terminus (Gu et al 2015).

### Administration of TrkB Inhibitors

The TrkB inhibitor may be administered as a composition comprising the inhibitor and one or more pharmaceutically acceptable carriers, adjuvants, diluents, and/or excipients, and may be administered by any route known in the art, including, but not limited to, orally, intravenously, intraperitoneally, intramuscularly, intrathecally, subcutaneously, sublingually, buccally, rectally, vaginally, ocularly, otically, nasally, by inhalation, by nebulization, topically, and transdermally. In certain embodiments, the inhibitor is administered intravenously or intraperitoneally.

One of skill in the art will be able to determine suitable dosing regimens in order to achieve the desired effect for a particular TrkB/PLCγ1 inhibitor, including period of administration, dose administered, and frequency of administration. In some embodiments, the inhibitor is administered for a period of about 1-4 days (including 2-3 days, 1 day, 2 days, or 3 days, etc.) or a period of about 2 weeks. In some embodiments the inhibitor is administered for a period of about 1 month. In some embodiments, the inhibitor is administered twice daily at a dose of or about 10-20 mg/kg.

One of skill in the art will understand that in certain embodiments of the invention, the TrkB inhibitor may be administered in combination with one or more antiseizure drugs, and when that is the case, the inhibitor and antiseizure drug(s) may be administered either sequentially or concurrently. The antiseizure drug(s) may be any antiseizure medication known in the art.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosures described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### EXAMPLES

### Example 1: Materials and Methods

### Animals

All animal procedures were approved by the Institutional Animal Care and Use Committee (IACUC) at Duke University and conform to the National Institutes of Health and Duke University institutional guidelines for the care and use of experimental animals. Animals were maintained on a 12-hour light/dark cycle with food and water available ad libitum. Wild type (WT) adult (8-12 wk) C57/bl6 male mice were obtained from Charles River. TrkB^{F616A} mice were originally obtained from Dr. David Ginty (Chen et al. 2005) and backcrossed to the C57/bl6 line for at least seven generations. This knockin mouse harbors a point mutation on the TrkB allele, substituting an alanine for phenylalanine within the ATP binding pocket of the TrkB kinase domain. This mutation renders TrkB protein uniquely susceptible to kinase inhibition by small molecule derivatives of the general kinase inhibitor PP1, including 1-(1,1-dimethylethyl)-3-(1-naphthalenylmethyl)-1H-pyrazolo[3,4-d]pyrimidin-4-amine (1NMPP1). Importantly, 1NMPP1 does not have any detectable effect in WT mice, and there are no differences in TrkB kinase activity in the TrkB^{F616A} compared to WT mice in the absence of this compound (Chen et al. 2005, Liu et al. 2013). Both male and female adult (8-12 wk) homozygous mice were used.

### Kindling

Video and EEG recordings were analyzed by a blinded, trained observer who quantified behavioral seizure class and duration of both electrographic and behavioral seizure.

Experiments were performed as previously described (He et al. 2010, He et al. 2014). A bipolar stimulating and recording electrode was inserted into the right amygdala (1.0 mm posterior, 2.9 mm lateral to bregma; 4.6 mm below dura). A skull screw was placed over the left frontal lobe as a ground electrode. After a postoperative recovery period of at least 5 days, animals were connected to a Grass Stimulator and monitored by both video and EEG. The current required to evoke an electrographic seizure (electrographic seizure threshold [EST]) was determined by applying a 1 s train of 1 msec biphasic-rectangular pulses at 60 Hz beginning at 20 μA. Additional stimulations were given in 20 μA increments at 1 min intervals until an electrographic seizure was detected. Animals then received two stimulations per day at the EST, with the behavioral seizure scores classified according to a modification of the Racine scale for mice (Borges et al., 2003; Racine, 1972): 0, normal activity; 1, arrest and rigid posture; 2, head nodding; 3, unilateral forelimb clonus; 4, rearing with bilateral forelimbs clonus; 5, rearing and falling; 6, tonic-clonic seizures with violent running and/or jumping. The criterion for “kindled” was the occurrence of three consecutive seizures of class 4 or greater, with limb clonus/tonus lasting at least 12 s.

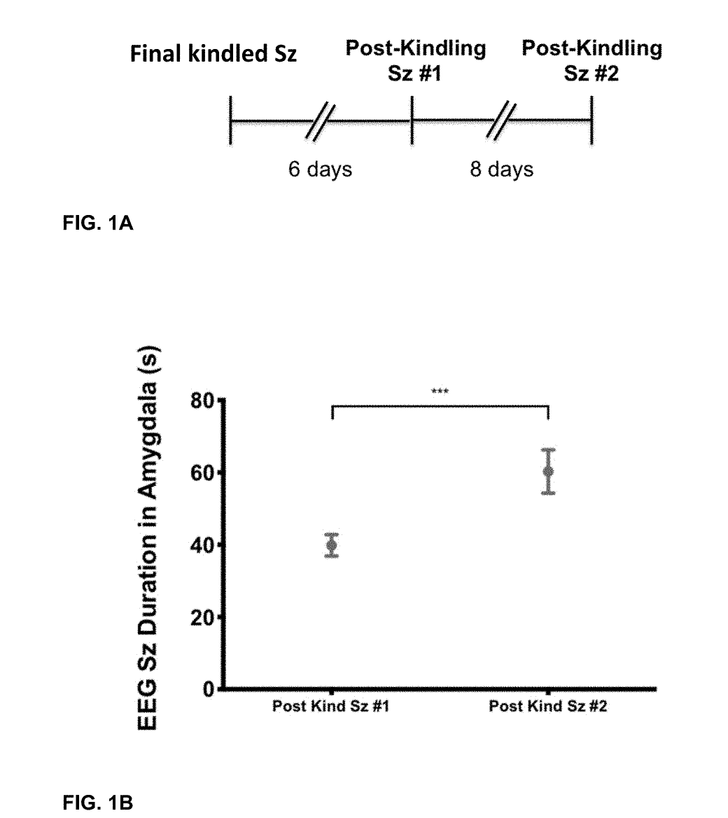

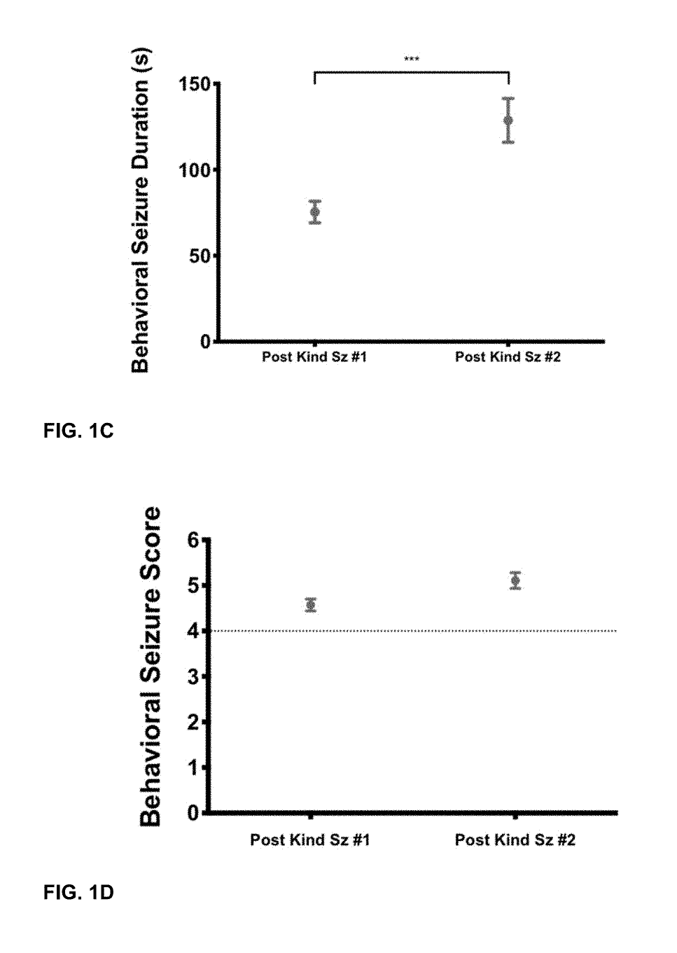

Design of individual experiments is presented in panel A of FIGS. 1-5. All “post-kindling seizures” (see FIG. 1A) were evoked with a stimulation intensity determined by assessing the EST a second time, typically 6 days after evoking the “final kindled seizure” the EST was determined by administering stimulations in 20 μA increments every 1 min, beginning at 20 μA, until an electrographic seizure was evoked. Notably, in experiments presented in FIGS. 1, 2, 3, and 5, experimental design mandated that the behavioral pattern accompanying the electrographic seizure of “Post-Kindling Seizure #1” was class 4 or greater.

### Treatments

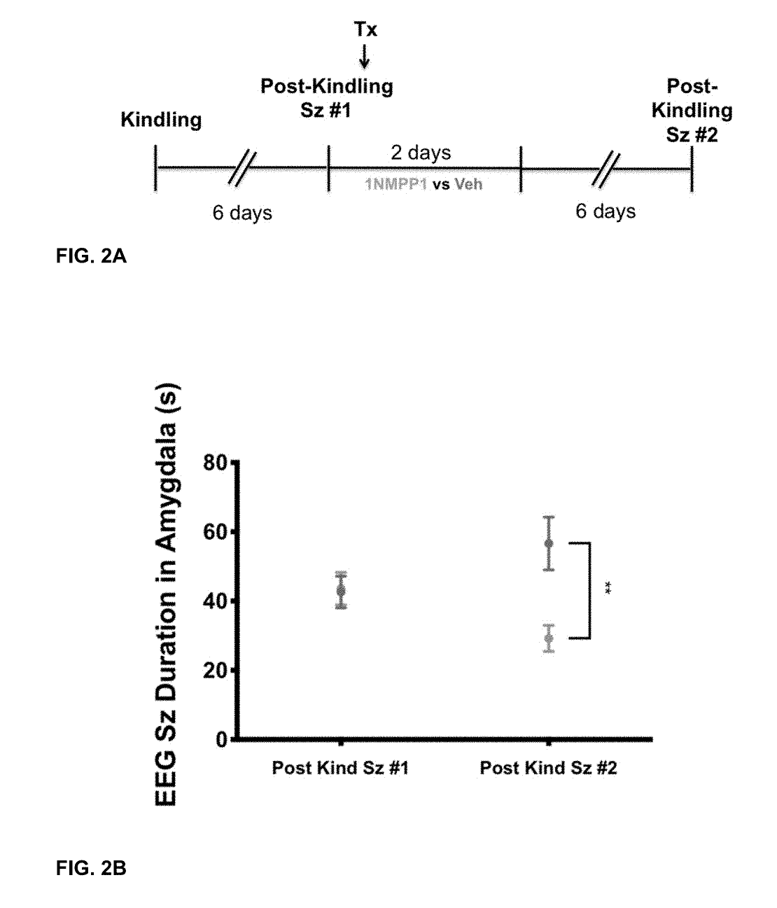

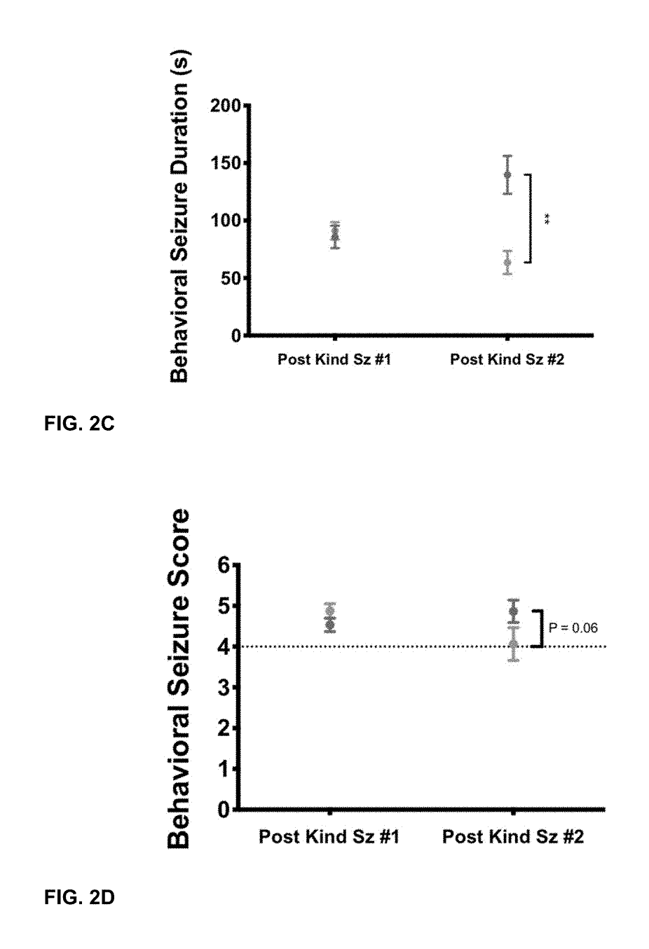

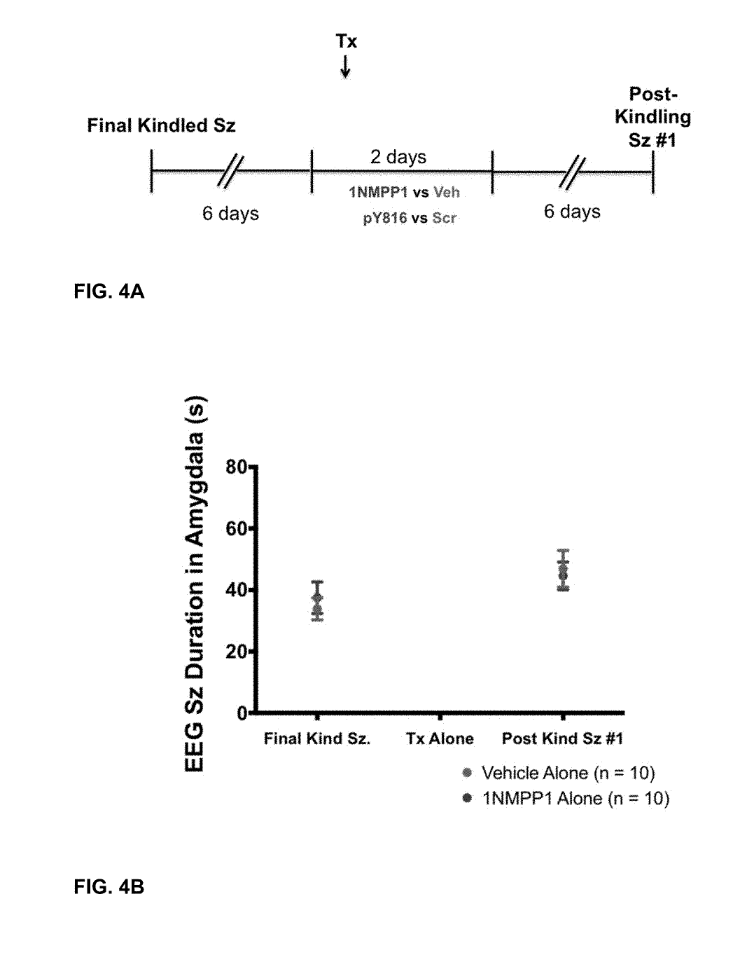

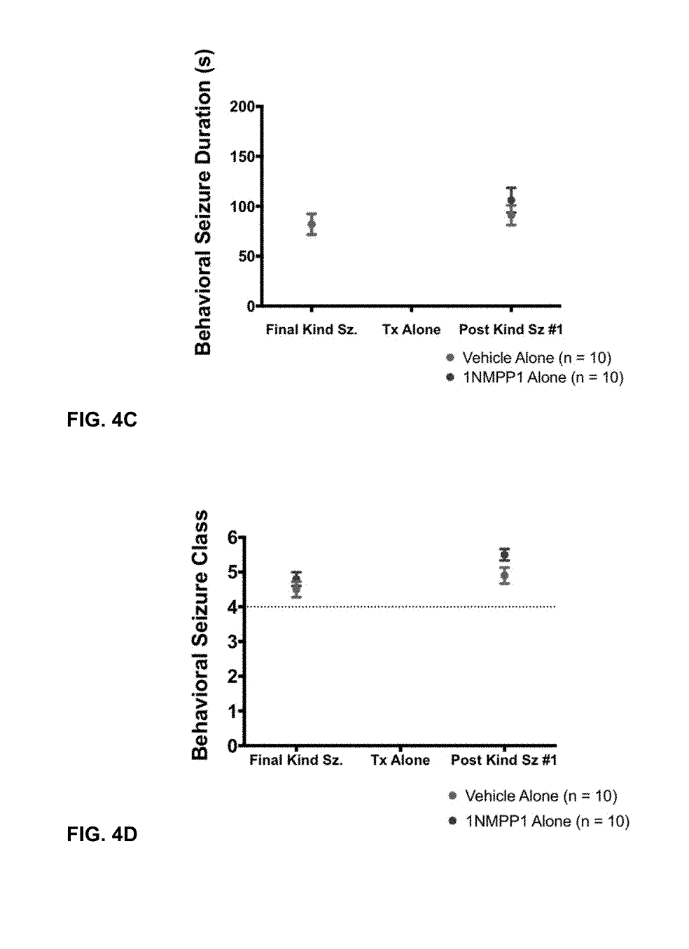

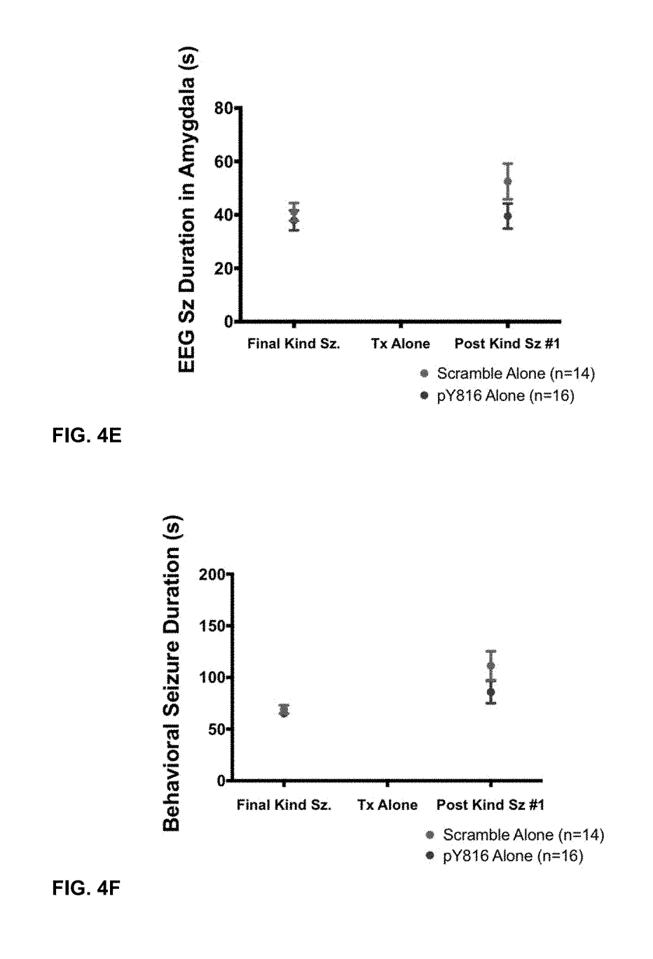

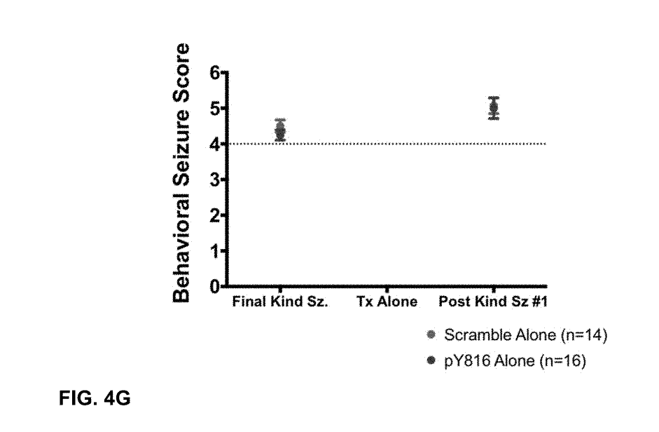

Prior to each administration, stock 100 mM 1NMPP1 was dissolved in a solubilization buffer containing 0.9% NaCL and 2.5% Tween-20 to a concentration of 1.67 mg/mL and dosed at 16.6 ug/g IP every 12 h for a total of five treatments. Vehicle injection served as a control. In addition, either 1NMPP1 (25 μM) or vehicle was included in drinking water for the 2 days of treatment (FIGS. 2 and 4).

Peptides were prepared as previously described (Gu et al. 2015). The sequence of human TrkB amino acids 807-820 (LQNLAKASPVpYLDI, SEQ ID NO:1) with the tyrosine at residue 817 phosphorylated (note that this corresponds to residue 816 in mouse TrkB protein) was conjugated at the N-terminus to the HIV trans-activating protein transduction domain (tat: YGRKKRRQRRR, SEQ ID NO: 2) to allow membrane permeability (termed “pY816”). The HIV tat sequence conjugated to a scrambled peptide (LVApYQLKIAPNDLS, SEQ ID NO: 3) served as a control (termed “Scr”). Peptides were synthesized and purified by Tufts Peptide Core Facility, dissolved in sterile PBS at 2 mg/mL, stored at −80° C., thawed just prior to treatment administration, and given at a dose of 20 mg/kg IP, for a total of five doses given 12 h apart. The quality of each batch of peptide was assessed by reverse phase HPLC verifying that at least 95% elutes as a single peak.

Carbamazepine (Sigma) was dissolved in a solubilization buffer of 2% Tween-80 and 70% propylene glycol at a concentration of 2 mg/mL and administered at a dose of 20 mg/kg, a dose of carbamazepine sufficient to produce therapeutic blood levels (Grabenstatter et al. 2007). Treatments were given every 4 h for two days. Solubilization buffer was used as a control.

### Statistical Analysis

All data analysis was performed by individuals blinded to treatment group and experimental condition. Animals were randomized to treatment groups immediately following administration of “Post-Kindling Seizure #1” and prior to any data analysis. Sample sizes were chosen based on power analysis. Unless otherwise stated, data are presented as mean±standard error of the mean (SEM). Unless otherwise stated, comparisons between two groups were analyzed using two-way ANOVA with repeated measures & post-hoc Bonferroni. A p<0.05 was considered significant.

### Example 2: Progression of Epileptogenesis Induced by an Isolated Seizure

We implemented a variation of the kindling model whereby a single evoked seizure induced increased duration of the next evoked electrographic and behavioral seizure, evidence of progression of epileptogenesis. To induce “kindling,” adult mice were subjected to repeated brief (1 second) low intensity stimulations locally within the amygdala twice daily, resulting in evoked seizures of increasing duration and propagation. Animals were termed “kindled” following the third consecutive evoked seizure with score of Class 4 or greater (the “Final Kindled Seizure”). Following a six-day stimulus free period, a series of stimuli (1 sec) was administered commencing at 20 μA and increasing by 20 μA at one minute intervals until an electrographic and behavioral seizure was evoked (Post-Kindling Seizure #1). Following an additional eight day stimulation free period, the current required to evoke Post-Kindling Seizure #1 was administered a second time and the duration of the resulting electrographic and behavioral seizure determined (Post-Kindling Seizure #2). The increased duration of the electrographic (FIG. 1B) and behavioral (FIG. 1C) features of Post-Kindling Seizure #2 compared to #1 provided a model whereby an isolated seizure caused increased duration of a subsequent seizure. Importantly, the temporal control of the occurrence of these seizures simplified determining whether a perturbation introduced following a seizure could limit progression.

### Example 3: Chemical-Genetic Inhibition of TrkB Kinase after an Evoked Seizure Prevents Progression

TrkB kinase is activated following an evoked seizure in the kindling model (He et al 2014). We therefore asked whether initiating inhibition of TrkB signaling immediately following an evoked seizure would prevent seizure-induced progression. We used the chemical-genetic approach with TrkB^{F616A} mice because this provides both molecular specificity and temporal control of inhibition of TrkB kinase activity. Initiating inhibition of TrkB kinase with 1NMPP1 immediately following Post-Kindling Seizure #1 in TrkB^{F616A} mice (FIG. 2A) significantly reduced the duration of both electrographic (FIG. 2B: 1NMPP1: 29.2±3.8 s, Vehicle: 56.6±7.6 s; p<0.01) and behavioral features of Post-Kindling Seizure #2 (FIG. 2C: 1NMPP1: 63.5±10 s, Vehicle: 139.8±16.5 s; p<0.01) when compared to vehicle-treated controls. A nonsignificant trend toward reduction of behavioral seizure class of Post-Kindling Seizure #2 was evident in 1NMPP1 treated animals (FIG. 2D).

Ascribing the effects of 1NMPP1 in the TrkB^{F616A} mice to inhibition of TrkB kinase requires that treatment of wild type mice with 1NMPP1 fails to reduce duration of Post-Kindling Seizure #2 in comparison to #1. Indeed, 1NMPP1 treatment of WT animals (n=9) following Post-Kindling Seizure #1 did not prevent increased duration of electrographic (Post-Kindling Sz #1: 35.1±5.3 s, Post-Kindling Sz #2: 67.4±13.5 s; p<0.01) or behavioral seizures (Post-Kindling Sz #1: 72.4±10.2 s, Post-Kindling Sz #2: 107.6±10.5 s; p<0.01).

### Example 4: pY816 Treatment after an Evoked Seizure Prevents Progression

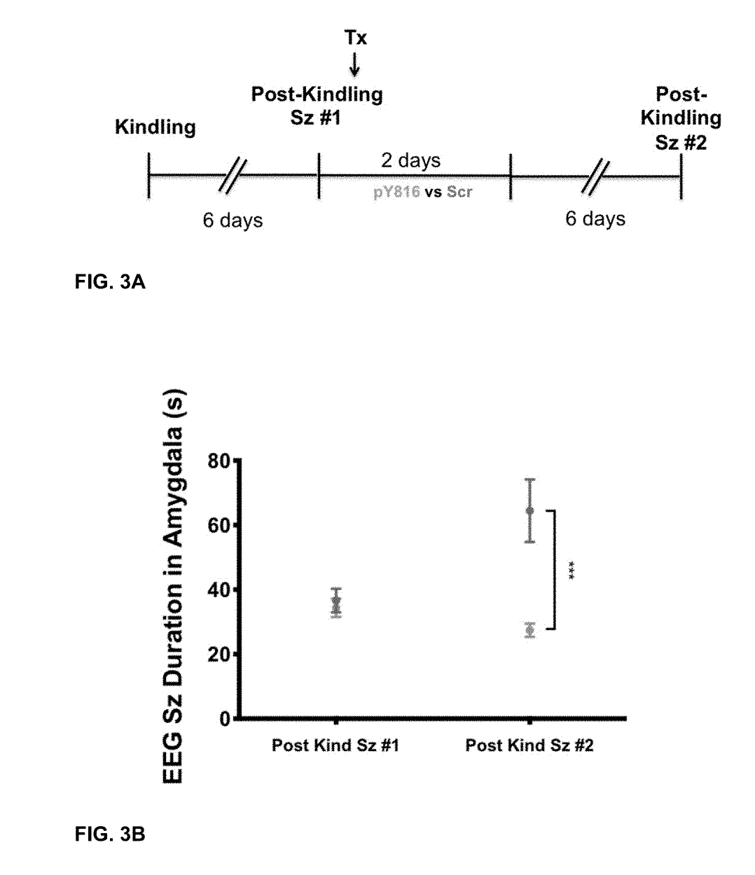

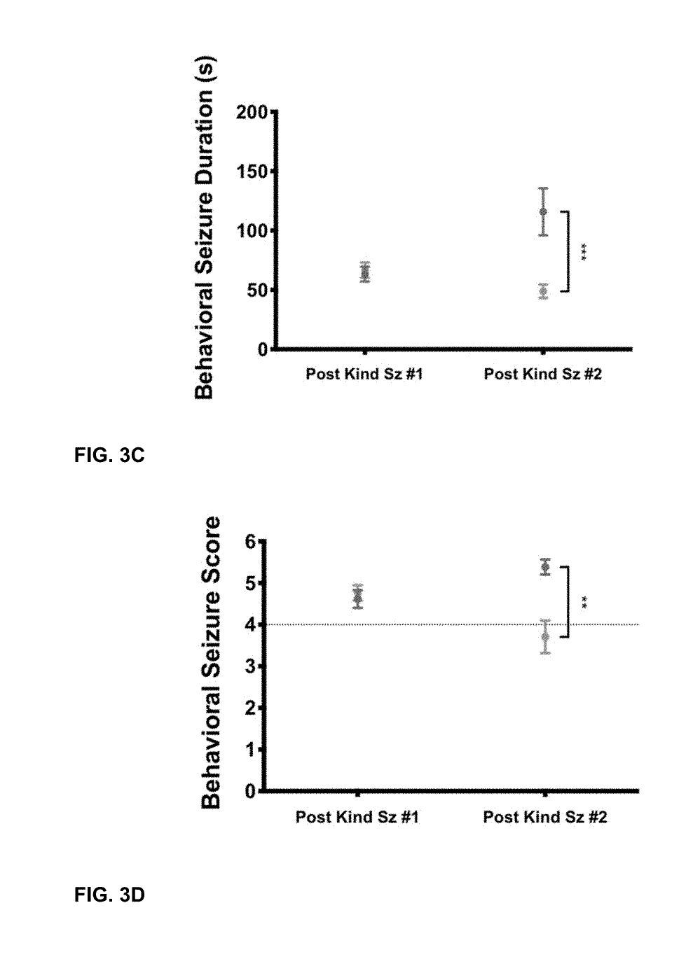

Our prior studies of status epilepticus-induced temporal lobe epilepsy implicated a causal role of a single signaling pathway downstream of TrkB, namely PLCγ1; this conclusion is based in part on the beneficial effects of pY816, a peptide that uncouples TrkB from PLCγ1 (Gu et al 2015). Importantly, an evoked seizure in the kindling model induced activation of PLCγ1 as assessed by a surrogate marker, the phosphorylation of PLCγ1 tyrosine 783 (He et al. 2014); moreover, treatment with pY816 immediately following repeated seizures inhibits activation of PLCγ1 (Gu et al 2015). Together these observations led us to ask whether treatment with pY816 following an isolated seizure prevented lengthening of subsequent seizure. Administration of pY816 peptide immediately following Post-Kindling Seizure #1 (FIG. 3A) reduced the duration of electrographic (FIG. 3B: pY816: 27.4±8.5, Scr: 64.5±9.8; p<0.001) and behavioral (FIG. 3C, pY816: 48.9±5.7, Scr: 115.8±19.8; p<0.001) features of Post-Kindling Seizure #2 in comparison to Scr controls (FIG. 3B-C). pY816 treatment also induced significant reductions of the class of Post-Kindling Seizure #2 in comparison to Scr controls (FIG. 3D: pY816: 3.7±0.4, Scr: 5.4±0.18; p<0.05).

### Example 5: Inhibition of TrkB-PLCγ1 Signaling Following an Evoked Seizure: Evidence of Reversion of Epileptogenesis to an Earlier Stage

Experiments described above demonstrate that transient inhibition of TrkB-PLCγ1 signaling following an evoked seizure limits progression of epileptogenesis as evident in comparisons of Post-Kindling Seizure #2 with #1 (FIGS. 2 and 3). Unexpectedly, this disruption also induced a reversion to an earlier stage of epileptogenesis evident in some, but not all, comparisons of Post-Kindling Seizure #2 with the Final Kindled Seizure. For example, inhibition of TrkB kinase produced a significant reduction in duration of electrographic seizure of Post-Kindling Seizure #2 compared to the Final Kindled Seizure (Post-Kindling Sz #2: 29.2±3.8, Final Kindled Sz: 43.8±4.5 s; p<0.05). Moreover, pY816 treatment produced a reduction of behavioral seizure class of Post-Kindling Seizure #2 compared to the Final Kindled Sz (Post-Kindling Sz #2: 3.7±0.3, Final Kindled Sz: 4.5±0.2 p<0.05). Furthermore, of the 33 animals receiving either 1NMPP1 or pY816 following Post-Kindling Sz #1, the seizure evoked with Post-Kindling Sz #2 was subconvulsive (less than Class 4) in 9 (4 treated with 1NMPP1 and 5 treated with pY816); by contrast, all but 1 control (vehicle or Scr treated, n=28) animal exhibited convulsive seizures of Class 4 or greater (p<0.05, Fisher's exact test).

### Example 6: Ineffectiveness of pY816 and 1NMPP1 in the Absence of a Preceding Evoked Seizure

The beneficial effects of fleeting inhibition of TrkB-PLCγ1 signaling following an evoked seizure led us to ask whether fleeting inhibition of TrkB-PLCγ1 signaling in the absence of a preceding evoked seizure alone is sufficient to limit severity of subsequent evoked seizures. To address this question, six days following the final kindled seizure, TrkB^{F616A} mice were treated with 1NMPP1 for two days (both via IP injection and in drinking water) and a seizure was evoked six days following cessation of 1NMPP1 (FIG. 4A). No differences were detected between 1NMPP1 and vehicle treated mice with respect to duration of electrographic (FIG. 4B) or behavioral (FIG. 4C) seizure or seizure class (FIG. 4D). The effects of pY816 in WT mice were investigated using a similar experimental design (FIG. 4A). No differences were detected between pY816 and Scr treated mice with respect to duration of electrographic (FIG. 4E) or behavioral (FIG. 4F) seizure or seizure class (FIG. 4G).

### Example 7: Treatment with Carbamazepine after an Evoked Seizure has No Effect on Subsequent Seizure Class or Duration

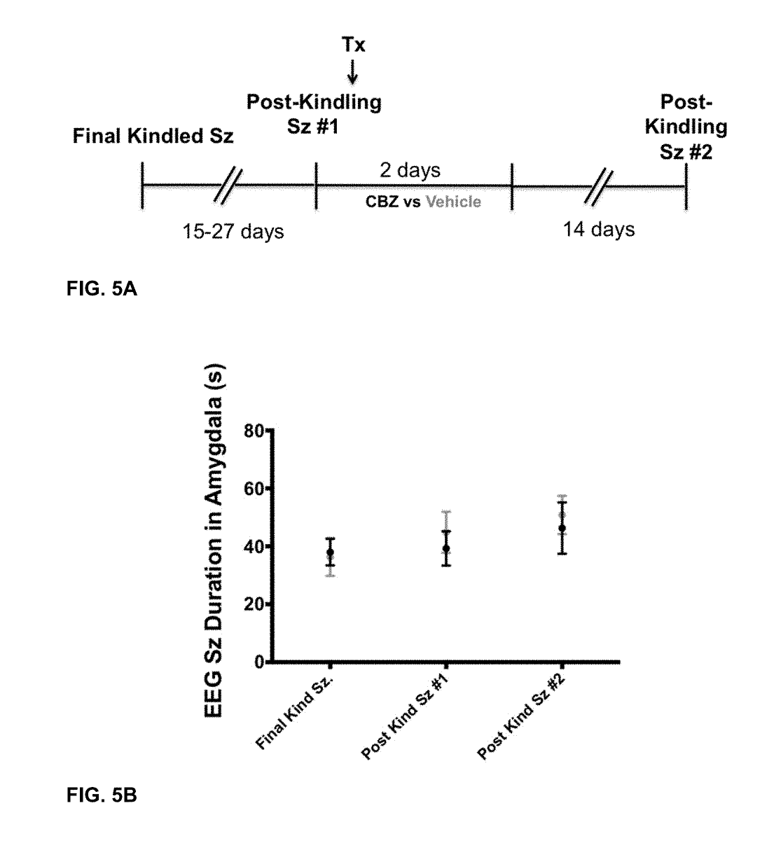

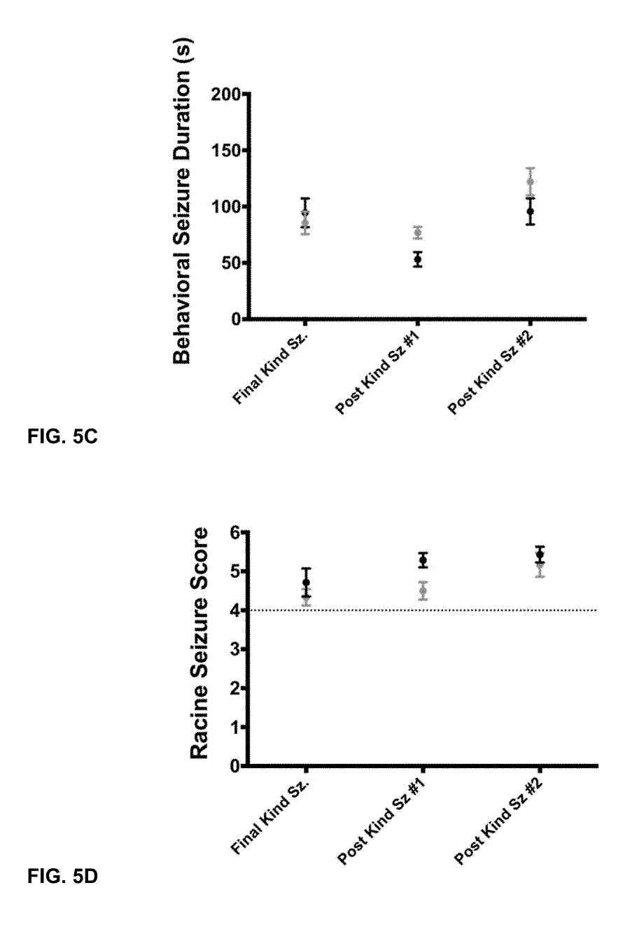

The beneficial effects of inhibitors of TrkB-PLCγ1 signaling administered transiently following a seizure raised the question as to whether a clinically effective antiseizure drug might have similar benefits. To address this question, a similar experimental design was employed whereby carbamazepine was administered for two days following an evoked seizure (FIG. 5A). Fifteen to twenty-seven days after the final kindled seizure, animals were treated with either carbamazepine (n=7, 20 mg/kg IP at four hour intervals for two days) or vehicle (n=6) following Post-Kindling Seizure #1. Fourteen days after the last dose, Post-Kindling Sz #2 was evoked. No differences were detected between carbamazepine and vehicle treated animals with respect to duration of electrographic (FIG. 5B) or behavioral (FIG. 5C) seizures or seizure class (FIG. 5D). The increased variability in seizure duration and score notwithstanding, these results demonstrate that treatment with a clinically effective anticonvulsant immediately following an evoked seizure is not sufficient to ameliorate seizure-induced progression in duration and seizure class of subsequent seizures.

### Example 8: Remission of Epilepsy Following Treatment with pY816

TrkB Kinase Inhibition after SE Prevents the Development of Epilepsy

Using a powerful chemical-genetic approach, we demonstrated that commencing TrkB kinase inhibition after SE and continuing it for just two weeks prevented both epilepsy and comorbid anxiety-like behavior when assessed many weeks later. (Liu et al., 2013). The importance was twofold: 1) it demonstrated that it was possible to intervene briefly following an insult and prevent SE-induced TLE; and 2) it identified a target for drug development.

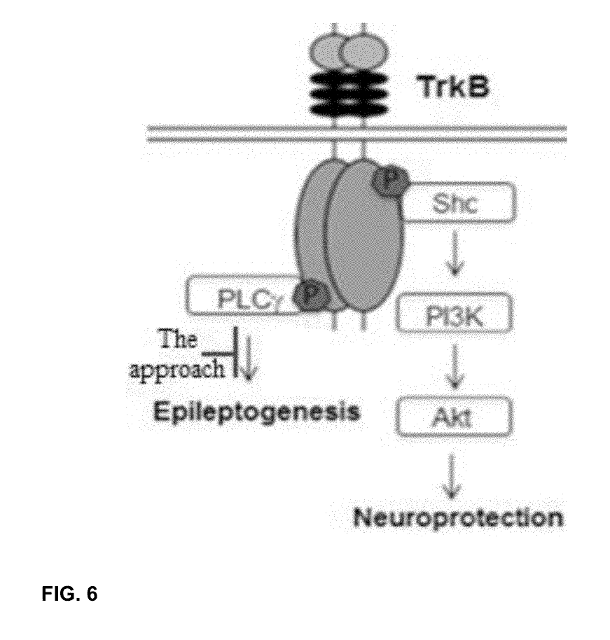

We subsequently identified the effector downstream of TrkB that promotes epileptogenesis, namely activation of phospholipase Cγ1 (PLCγ1) (FIG. 6). (Gu et al., 2015). This insight enabled us to design a drug to prevent epilepsy in this animal model. We designed a novel peptide that uncouples TrkB from PLCγ1, termed pY816. Corresponding to amino acids 806-819 of human TrkB in which Y816 is phosphorylated, pY816 can bind the SH2 domain of PLCγ1 and prevent binding and activation of PLCγ1 by endogenous TrkB. To promote pY816 permeation of cell membranes and the blood-brain barrier, we fused the protein transduction domain of the viral TAT protein to the N terminus of pY816 and used an HIV-1 Tat conjugated randomly scrambled peptide (Scr) as a negative control. We demonstrated that treatment with pY816, initiated after status epilepticus and continued for just two days, reduced the number of recurrent seizures by 90% when assayed 1-2 months later. (Gu et al., 2015). Importantly, the amino acids 806-819 of TrkB contained in pY816 are identical in mouse and human, strengthening the likelihood that pY816 itself can be used to treat humans. The significance of these findings was twofold: 1) it demonstrated that briefly inhibiting TrkB-induced activation of PLCγ1 following an insult prevents SE-induced TLE; and 2) it identified TrkB and PLCγ1 as targets for drug development.

Subsequent studies (Examples 2-6 above) demonstrated that TrkB-PLCγ1 inhibitors prevented the increased duration of electrographic and behavioral seizures induced by an isolated seizure. Remarkably, a subset of animals exhibited a reversion of epileptogenesis to an earlier state (Example 5 above), leading us to the idea that inhibitors of TrkB and PLCγ1 might induce a remission of epilepsy in animals that had already become epileptic. Importantly, hippocampi surgically isolated from patients with medically refractory TLE exhibit striking increases of BDNF mRNA and protein (Mathern et al., 1997; Murray et al., 2000), raising the possibility that enhanced TrkB signaling contributes to the persistence and medical refractoriness of seizures in these patients. We therefore set out to assess whether brief treatment with pY816 after onset of epilepsy might reduce the occurrence of spontaneous recurrent seizures (SRS) during or following treatment.

Dose Dependent Inhibition of PLCγ1 by Intraperitoneal Administration of pY816

Determining whether pY816 may be beneficial in epileptic animals required modification of our study of the kainic acid (KA)-status epilepticus (SE) model. To more closely mimic the clinical scenario in which patients seek medical attention for established TLE, we induced KA-SE and then conducted video-EEG recordings to detect spontaneous recurrent seizures during baseline periods of two to four weeks until they developed TLE prior to treating with pY816 peptide. In contrast to just three days of treatment with pY816 deployed in previous studies, we sought a more prolonged treatment with pY816 for the current experiments, namely two weeks. The intravenous route of administration used for three days in the prior studies was not feasible for two weeks, leading us to ask whether treatment via intraperitoneal route could inhibit activation of PLCγ1. We used the phosphorylation of residue Y783 of PLCγ1 detected on western blot of brain lysates as a surrogate measure of PLCγ1 activation.

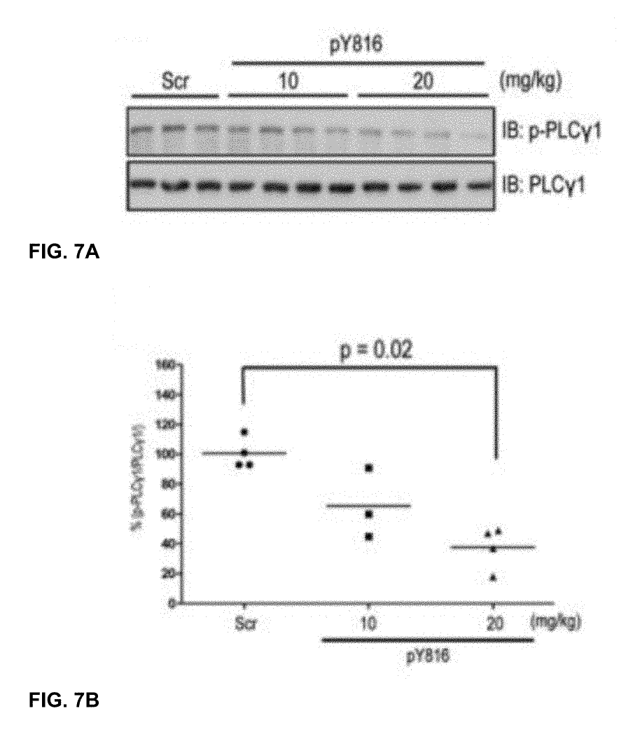

Treatment of adult mice with a single dose of pY816 three hours prior to death revealed a dose dependent inhibition of PLCγ1 activation, inhibition approximating 60% with a dose of 20 mg/kg as shown in FIG. 7, where either Scr or pY816 (10 or 20 mg/kg, i.p.) was injected intraperitoneally into wild type mice and animals were euthanized 3 hours later. Hippocampal lysates (30-40 μg) were subjected to SDS_PAGE and western blotting. The blots were probed with antibodies specifically recognizing p-PLCγ1 (pY783) (a surrogate for activation of PLCγ1) and PLCγ1. In comparison to Scr control, ratios of immunoreactivity of p-PLCγ1 (pY783) to PLCγ1 showed that pY816 (20 mg/kg) significantly reduces p-PLCγ1 (pY783) at 3 h (˜60%).

Importantly, the extent of this inhibition is similar to that of single intravenous injection of pY816.

Remission of Epilepsy Following Treatment with pY816

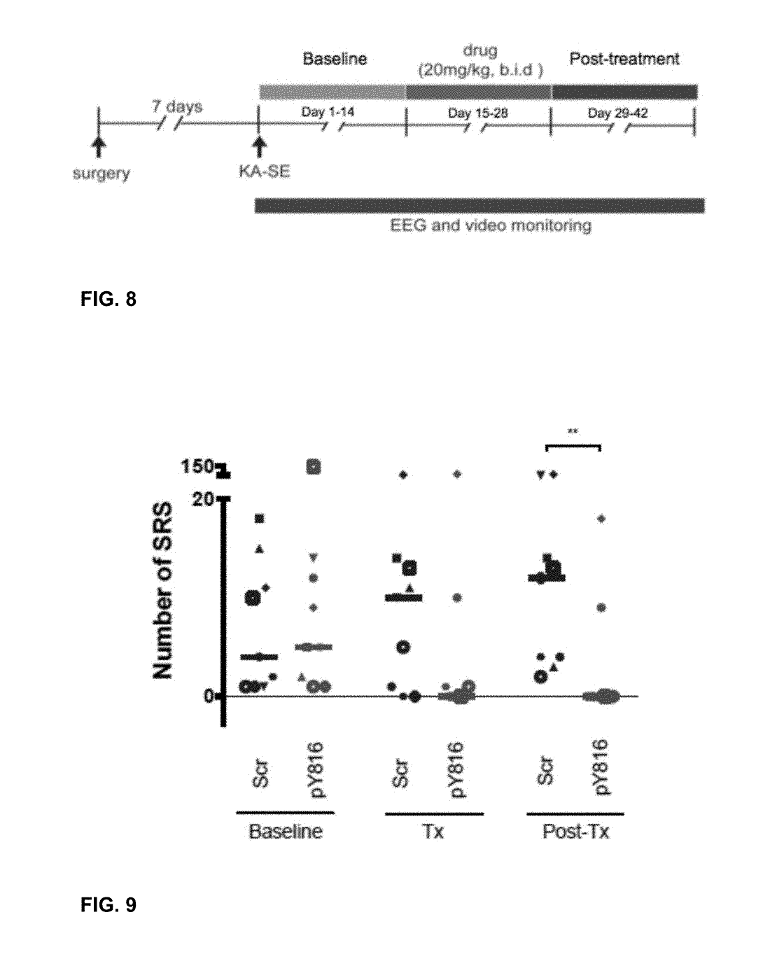

Having verified that intraperitoneal administration of pY816 inhibited PLCγ1 activation, we then asked whether treatment with pY816 initiated after onset of epilepsy and continued for two weeks would reduce the severity of epilepsy or even induce a remission. Towards that end, we induced KA-SE but then followed the animals for several weeks until they developed TLE as assessed by detection of SRS in continuous video-EEG recordings. We quantified the number of SRS in each animal by blinded review of video-EEG recordings during three distinct two week epochs (FIG. 8): a) baseline; b) treatment with pY816 (n=9) or Scr control (n=6), 20 mg/kg IP twice daily for two weeks (on Tx); c) following termination of treatment (Post Tx). As noted in FIG. 8, following induction of status epilepticus induced by infusion of kainic acid into amygdala (Gu et al 2015) animals underwent continuous video-EEG recording to detect spontaneous recurrent seizures. Following baseline recording of two weeks, animals were treated with either pY816 or Scr control peptide (20 mg/kg) twice daily for two weeks (Day 15-28). Video-EEG recording continued for two weeks following termination of treatment (Post-treatment).

Results are shown in FIG. 9, where each symbol represents the number of seizures experienced by a given animal during Baseline, Treatment (Tx), or Post-treatment (Post Tx) periods. Horizontal bars designate median. Treatment (Tx) consisted of either pY816 or Scr control given 20 mg/kg twice daily for two weeks. Note that seizure detection was performed by analyses of video-EEG recordings by two blinded investigators.

A modest reduction in the number of seizures was evident during the two weeks of treatment with pY816 (FIG. 9). Remarkably, SRS was reduced during the two weeks following termination of treatment. That is, in contrast to Scr treatment in which all 9 animals exhibited SRS, pY816 treatment rendered 7 of 9 animals free of SRS during the two weeks following termination of treatment (Fisher's exact test p=0.0023, two-tailed). No overt untoward effects were detected in pY816 or Scr control treated animals during or following treatment. SRS occurring in animals following SE typically exhibit responsiveness to anticonvulsants similar to patients with medically refractory TLE, with recurrent seizures persisting at doses without unwanted effects. (Murray et al 2000).

Treatment with Carbamazepine does not Induce Remission of TLE

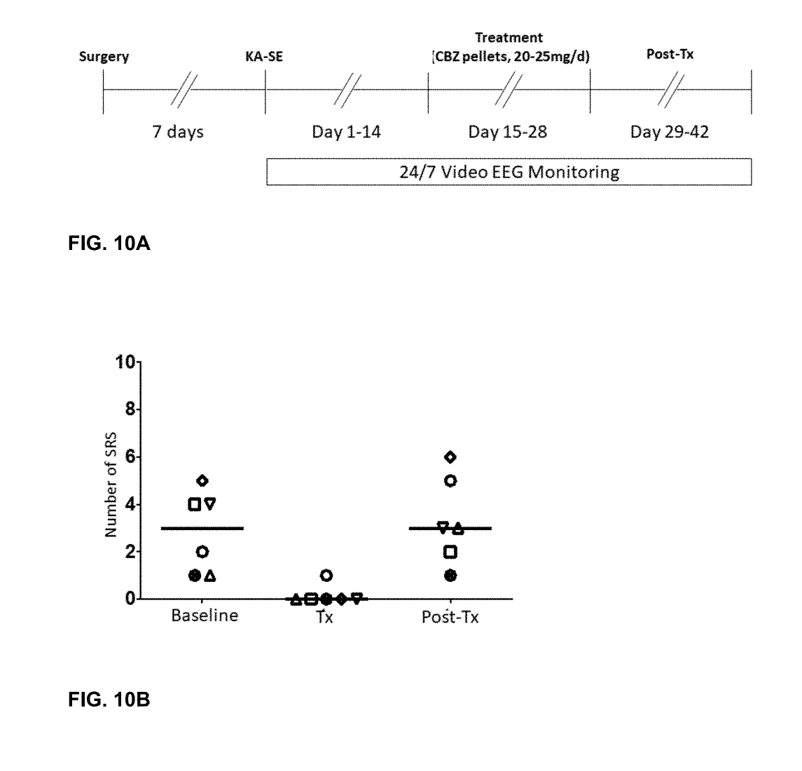

Antiseizure drugs in current clinical use provide symptomatic relief of seizures in many but not all patients, but seizures typically recur upon cessation of the drug. To verify that similar findings occur in our animal model, we tested the effect of an antiseizure drug in current clinical use in our model (carbamazepine). We infused additional animals with KA and assessed the occurrence of SRS with continuous video-EEG monitoring for several weeks. Six animals exhibited at least one SRS during this baseline period and were administered carbamazepine pellets, a commonly used anticonvulsant for treatment of TLE, for two weeks at a dose of 800 mg/kg/day. Carbamazepine treatment effectively suppressed SRS in all but one animal, which had a single seizure while on treatment (FIG. 10). Importantly, seizures recurred following cessation of treatment in each of the six animals, typically at a frequency similar to that seen in the baseline period (Figure **). Thus, like TLE in humans, continuous treatment with an antiseizure drug suppresses seizures, yet seizures recur upon discontinuation of treatment.

### SUMMARY

The induction of a remission of TLE by fleeting treatment with pY816 stands in stark contrast to treatments in current clinical use for this disorder. These findings raise the possibility that fleeting treatment with pY816 may reduce the severity of epilepsy in patients with temporal lobe epilepsy. This could be evidenced by enhanced response to conventional antiseizure drugs, thereby transforming medically refractory to medically responsive epilepsy. Alternatively, induction of remission of epilepsy, even if temporary, could improve the quality of patients' lives of many afflicted with this dreadful disorder.

### REFERENCES

Annegers J F, Hauser W A, Shirts S B, and Kurland L T (1987). Factors prognostic of unprovoked seizures after febrile convulsions. NEJM 316: 493-498.
Bailey, Justin J., et al. “Tropomyosin receptor kinase inhibitors: an updated patent review for 2010-2016—Part I.” Expert Opinion on Therapeutic Patents, 27:6, 733-751 (2017).
Barker-Haliski, Melissa L., et al. “Disease modification in epilepsy: from animal models to clinical applications.” Drugs 75.7 (2015): 749-767,
Bernasconi, Neda, Jun Natsume, and Andrea Bernasconi. “Progression in temporal lobe epilepsy Differential atrophy in mesial temporal structures.” Neurology 65.2 (2005): 223-228.
Bernhardt, Boris C., of al. “Longitudinal and cross-sectional analysis of atrophy in pharmacoresistant temporal lobe epilepsy.” Neurology 72.20 (2009): 1747-1754.
Bertram, Edward H., and John Cornett, “The ontogeny of seizures in a rat model of limbic epilepsy: evidence for a kindling process in the development of chronic spontaneous seizures.” Brain research 625.2. (1993): 295-300.
Binder, D. K., Routbort, M. J. and McNamara, J. O., 1999. Immunohistochemical evidence of seizure-induced activation of trk receptors in the mossy fiber pathway of adult rat hippocampus. Journal of Neuroscience, 19(11), pp. 4616-4626.
Bonin, R. P. and De Koninck, Y., 2014. A spinal analog of memory reconsolidation enables reversal of hyperalgesia. Nature neuroscience, 17(8), p. 1043.
Boon, Paul, et al. “A prospective, multicenter study of cardiac-based seizure detection to activate vagus nerve stimulation.” Seizure 32 (2015): 52-61.
Chen, Zhibin, et al. “Treatment outcomes in patients with newly diagnosed epilepsy treated with established and new antiepileptic drugs: a 30-year longitudinal cohort study.” JAW neurology 75.3 (2018): 279-286.
Coan, A. C., et al. “Seizure frequency and lateralization affect progression of atrophy in temporal lobe epilepsy.” Neurology 73.11 (2009): 834-842.
Danzer, S. C., He, X., Loepke, A M. and McNamara, J. O., 2010. Structural plasticity of dentate granule cell mossy fibers during the development of limbic epilepsy. Hippocampus, 20(1), pp. 113-124.
Engel J, Williamson P D, and Wieser H-G (1998). Mesial temporal lobe epilepsy. In Epilepsy: A Comprehensive Textbook, Lipponcott-Raven, Philadelphia, Chapter 231: 2417-2426.
Fonseca, Rosanna, U. Valentin Nägerl, and Tobias Bonhoeffer. “Neuronal activity determines the protein synthesis dependence of long-term potentiation.” Nature neuroscience 9.4 (2006): 478.
French J A, Williamson P D, Thadani V M, Darcey T M, Mattson R H, Spencer S S, and Spencer D D (1993). Characteristics of medial temporal lobe epilepsy: I. Results of history and physical examination. Ann Neurol 34: 774-780.
Goddard, 1967. Development of epileptic seizures through brain stimulation at low intensity. Nature, 214(5092), p. 1020.
Goddard, G. V., McIntyre, D. C. and Leech, C. K., 1969. A permanent change in brain function resulting from daily electrical stimulation. Experimental neurology, 25(3), pp. 295-330.
Gowers, W. R. “Epilepsy and other chronic convulsive diseases. vol. 1, p. 6,” London: Williams Wood (1881).
Grabenstatter, Heidi L., Suzanne Clark, and F. Edward Dudek. “Anticonvulsant effects of carbamazepine on spontaneous seizures in rats with kainite-induced epilepsy: comparison of intraperitoneal injections with drug-in-food protocols.” Epilepsia 48.12 (2007): 2287-2295.
Gu B, Huang Y Z, He X P, Joshi R B, Jang W, McNamara J O. A peptide uncoupling BDNF receptor TrkB from Phospholipase Cγ1 prevents epilepsy induced by status epilepticus. Neuron 88:1-8, 2015.
Hauser, W. Allen, and Ju R. Lee. “Do seizures beget seizures?.” Progress in brain research. Vol. 135, Elsevier, 2002. 215-219.
Hauser, W. Allen. “Questioning the Effectiveness of Newer Antiseizure Medications.” JAMA neurology 75.3 (2018): 273-274.
Helgager, Jeffrey, Gumei Liu, and James O. McNamara. “The cellular and synaptic location of activated TrkB in mouse hippocampus during limbic epileptogenesis.” Journal of Comparative Neurology 521.3 (2013): 499-521.
Hellier, Jennifer L., et al. “Recurrent spontaneous motor seizures after repeated low-dose systemic treatment with kainate: assessment of a rat model of temporal lobe epilepsy.” Epilepsy research 31.1 (1998): 73-84.
Huang, Yang Z., et al. “Zinc-mediated transactivation of TrkB potentiates the hippocampal mossy fiber-CA3 pyramid synapse.” Neuron 57.4 (2008): 546-558.
Kadam, Shilpa D., et al. “Continuous electroencephalographic monitoring with radio-telemetry in a rat model of perinatal hypoxia-ischemia reveals progressive post-stroke epilepsy.” Journal of Neuroscience 30.1 (2010): 404-415,
Kang, H. and Schuman, E. M., 1995. Long-lasting neurotrophin-induced enhancement of synaptic transmission in the adult hippocampus. Science, 267(5204), pp. 1658-1662.
Liu G, Gu B, He X P, Joshi R B, Wackerle H D, Rodriguiz R M, Wetsel W C, and McNamara J O (2013). Transient inhibition of TrkB kinase after status epilepticus prevents development of temporal lobe epilepsy. Neuron 79: 31-38.
Mathern G W, Babb T L, Micevych P E, Blanco C E, Pretorius J K (1997) Granule cell mRNA levels for BDNF, NGF, and NT-3 correlate with neuron losses or supragranular mossy fiber sprouting in the chronically damaged and epileptic human hippocampus. Mol Chem Neuropathol 30(1-2):53-76.
Misanin, James R., Ralph R. Miller, and Donald J. Lewis. “Retrograde amnesia produced by electroconvulsive shock after reactivation of a consolidated memory trace.” Science 160.3827 (1968): 554-555,
Murray K D, Isackson P J, Eskin T A, King M A, Montesinos S P, Abraham L A, Roper S N. (2000) Altered mRNA expression for brain-derived neurotrophic factor and type II calcium/calmodulin-dependent protein kinase in the hippocampus of patients with intractable temporal lobe epilepsy. J Comp Neurol. 418(4):411-22.
Nader, K., Schafe, G. E. and Le Doux, J. E., 2000, Fear memories require protein synthesis in the amygdala for reconsolidation after retrieval. Nature, 406(6797), p. 722.
Pinel, John P J, and Louis 1. Rovner. “Experimental epileptogenesis: kindling-induced epilepsy in rats.” Experimental neurology 58.2 (1978): 190-202.
Pitkanen A (2010). Therapeutic approaches to epileptogenesis—hope on the horizon. Epilepsia 51(S3): 2-17.
Racine, Ronald J. “Modification of seizure activity by electrical stimulation: I. After-discharge threshold.” Electroencephalography and clinical neurophysiology 32.3 (1972): 269-279.
Racine, Ronald, Larry Tuff, and Josef Zaide. “Kindling, unit discharge patterns and neural plasticity.” Canadian Journal of Neurological Sciences 2.4 (1975): 395-405.
Represa, A., La Salle, G. L. G. and Ben-Ari, Y., 1989. Hippocampal plasticity in the kindling model of epilepsy in rats. Neuroscience letters, 99(3), pp. 345-350.
Schildt, S., Endres, T., Lessmann, V. and Edelmann, E., 2013. Acute and chronic interference with BDNF/TrkB-signaling impair LTP selectively at mossy fiber synapses in the CA3 region of mouse hippocampus. Neuropharmacology, 71, pp. 247-254.
Shoeb, Ali, et al. “Non-invasive computerized system for automatically initiating vagus nerve stimulation following patient-specific detection of seizures or epileptiform discharges.” International Journal of Neural Systems 19.03 (2009): 157-172.
Stoop, R. and Poo, M. M., 1996. Synaptic modulation by neurotrophic factors: differential and synergistic effects of brain-derived neurotrophic factor and ciliary neurotrophic factor. Journal of Neuroscience, 16(10), pp. 3256-3264.
Sutula, T. and Steward, O., 1987. Facilitation of kindling by prior induction of long-term potentiation in the perforant path. Brain research, 420(1), pp. 109-117.
Tsai M H, Chuang Y C, Chang H W, Chang W N, Lai S L, Huang C R, Tsai N W, Wang H C, Lin Y J, and Lu C H (2009). Factors predictive of outcome in patients with de novo status epilepticus. QJM 102: 57-62.
White H S, Löscher W (2014) Searching for the ideal antiepileptogenic agent in experimental models: single treatment versus combinatorial treatment strategies. Neurotherapeutics 11(2):373-84.
Williams, Philip A., et al. “Development of spontaneous recurrent seizures after kainate-induced status epilepticus,” Journal of Neuroscience 29.7 (2009): 2103-2112.

We claim:

1. A method of treating a subject susceptible to the development of epilepsy comprising administering to the subject a therapeutic amount of a receptor tyrosine kinase B (TrkB) inhibitor.

2. The method of claim 1, wherein the treating comprises limiting the progression of epileptogenesis.

3. The method of claim 1, wherein the treating comprises the reversion of epileptogenesis to an earlier stage.

4. The method of claim 1, wherein the TrkB inhibitor is administered to the subject immediately following termination of an isolated seizure.

5. The method of claim 4, wherein the isolated seizure is not status epilepticus (SE).

6. The method of claim 4, wherein subsequent seizures do not occur or are not of increased severity and/or duration.

7. The method of claim 1, wherein the TrkB inhibitor is administered at a dose of about 10-20 mg/kg.

8. The method of claim 4, wherein the TrkB inhibitor is administered for 1-4 days.

9. The method of claim 1, wherein the TrkB inhibitor is a phospholipase Cγ1 (PLCγ1) inhibitor.

10. The method of claim 6, wherein the PLCγ1 inhibitor is pY816.

11. A method of inducing remission of epilepsy in a subject comprising administering a therapeutically effective amount of a TrkB inhibitor or a PLCγ1 inhibitor.

12. The method of claim 11 wherein the subject is suffering from temporal lobe epilepsy (TLE).

13. The method of claim 12 wherein the subject is suffering from medically refractory TLE.

14. The method of claim 11 wherein the PLCγ1 inhibitor is pY816.

15. The method of claim 11 wherein the inhibitor is administered for a period of about 2 weeks.

16. The method of claim 11 wherein the inhibitor is administered intravenously or intraperitoneally.

17. The method of claim 11 wherein the inhibitor is administered twice daily at a dose of about 10-20 mg/kg.

18. A method of transforming medically refractory epilepsy in a subject to medically responsive epilepsy comprising administering a therapeutically effective amount of a TrkB inhibitor or a PLCγ1 inhibitor, optionally in combination with one or more antiseizure drugs.

19. The method of claim 18 wherein the PLCγ1 inhibitor is pY816.

20. The method of claim 18 wherein the inhibitor is administered for a period of about 2 weeks.


SEQUENCE LISTING (3 sequences)

SEQ ID NO: 1 - protein, 14 aa, Artificial Sequence
  Synthetic peptide
  MISC_FEATURE (11)..(11): Tyrosine is phosphorylated
LQNLAKASPVYLDI

SEQ ID NO: 2 - protein, 11 aa, Artificial Sequence
  Synthetic peptide
YGRKKRRQRRR

SEQ ID NO: 3 - protein, 14 aa, Artificial Sequence
  Synthetic peptide
  MISC_FEATURE (4)..(4): Tyrosine is phosphorylated
LVAYQLKIAPNDLS